(19) United States
(12) Patent Application Publication
(10) Pub. No.: US 2025/0331739 A1
(43) Pub. Date: Oct. 30, 2025

(54) Estimation Method, Estimation Program, Estimation System, Determination Method, and Estimation Marker

(71) Applicant: SHIMADZU CORPORATION, Kyoto-shi (JP)

(72) Inventor: Mikihiro YAMANAKA, Kyoto-shi (JP)

(21) Appl. No.: 18/873,528

(22) PCT Filed: May 30, 2023
(86) PCT No.: PCT/JP2023/020060

§ 371 Date: Mar. 26, 2025

(30) Foreign Application Priority Data
Jun. 15, 2022 (JP) 2022-096166
Mar. 17, 2023 (JP) 2023-042917

(51) Int. Cl. A61B 5/145; A61B 5/00; G16H 50/70

(52) U.S. Cl. CPC A61B 5/14532; A61B 5/7246; A61B 5/742; G16H 50/70

(57) ABSTRACT

An estimation method includes: obtaining a measurement value of advanced glycation end products of a subject; and estimating, using a correlation between a measurement value of advanced glycation end products and a blood glucose spike frequency prepared in advance, a blood glucose spike frequency of the subject based on the measurement value of advanced glycation end products of the subject obtained in the obtaining.

### TECHNICAL FIELD

The present disclosure relates to an estimation method, an estimation program, and an estimation system for estimating a blood glucose spike frequency of a subject, a determination method for determining a blood glucose spike frequency of a subject, and an estimation marker.

### BACKGROUND ART

One of the substances that cause aging is advanced glycation end products (hereinafter also referred to as “AGEs”). The AGEs are a generic term for a plurality of compounds, and are generated by saccharides and proteins that bind to each other and react by oxidation, condensation, and dehydration. The AGEs are believed to accumulate in the body due to disordered lifestyle habits such as eating habits, exercise habits, sleep habits, fever or inflammation to injury, and stress, and cause lifestyle-related diseases (e.g., diabetes, dementia) or age-related diseases.

PTL 1 discloses a sensor that receives fluorescence excited by light applied to the skin of a subject and measures a degree of accumulation of AGEs based on the intensity of the received fluorescence. Since AGEs generally change in several weeks although there are individual differences, a subject measures the AGEs at a frequency of, for example, once in several weeks.

As a method of checking to see if lifestyle habits, in particular dietary habits, are disordered, it is known to continuously measure a blood glucose level (hereinafter, a glucose level in interstitial fluid that exhibits approximately the same behavior as the blood glucose level is also referred to as the “blood glucose level” for convenience) over a certain period of time (e.g., two weeks). The blood glucose level is indicated by the amount of glucose in the blood, and may be significantly influenced by the types or amounts of nutrients contained in a meal. The blood glucose level can vary rapidly, such as sharply rising to the same level as that of a diabetes patient followed by a sharp drop shortly thereafter in a postprandial state, while being similar to that of a healthy individual in a fasting state. Such rapid variation in the blood glucose level is also referred to as a “blood glucose spike.” It has been reported that the occurrence of the blood glucose spikes is a factor that causes diseases such as arteriosclerosis, dementia, or cancer, and those who experience the blood glucose spikes need to review their lifestyle habits in order to prevent lifestyle-related diseases or age-related diseases.

PTL 2 discloses a sensor that performs intermittently scanned CGM (is-CGM) of continuously monitoring a blood glucose level over a certain period of time by inserting a measuring needle placed on a sensor unit under the skin and the like.

### CITATION LIST

### Patent Literature

PTL 1: Japanese Patent Laying-Open No. 2015-223431
PTL 2: Japanese Patent Laying-Open No. 2011-212118

### SUMMARY OF INVENTION

### Technical Problem

By performing continuous glucose monitoring, a subject can check whether or not blood glucose spikes have occurred, and if so, a frequency of the blood glucose spikes (hereinafter also referred to as a “blood glucose spike frequency”), and is motivated to review his/her lifestyle habits. In the case where the continuous glucose monitoring is performed, however, it is important to keep the continuous glucose monitoring to a necessary minimum because the subject goes about his/her daily life with a measuring needle inserted under the skin and the like for a certain period of time. The continuous glucose monitoring is also costly. Therefore, the continuous glucose monitoring is more burdensome to the subject and tends to be avoided by the subject, as compared to measurement of AGEs, which only needs to be done at a frequency of once a week or once in several weeks.

The present disclosure has been made to solve the above-described problem, and an object thereof is to provide a technique for recognizing a blood glucose spike frequency while suppressing a burden on a subject.

### Solution to Problem

An estimation method according to an aspect of the present disclosure estimates a blood glucose spike frequency of a subject by a computing device. The estimation method includes, as processing to be performed by the computing device: obtaining a measurement value of advanced glycation end products of the subject; and estimating, using a correlation between a measurement value of advanced glycation end products and a blood glucose spike frequency prepared in advance, a blood glucose spike frequency of the subject based on the measurement value of advanced glycation end products of the subject obtained in the obtaining.

An estimation program according to another aspect of the present disclosure estimates a blood glucose spike frequency of a subject. The estimation program causes a computing device to perform: obtaining a measurement value of advanced glycation end products of the subject; and estimating, using a correlation between a measurement value of advanced glycation end products and a blood glucose spike frequency prepared in advance, a blood glucose spike frequency of the subject based on the measurement value of advanced glycation end products of the subject obtained in the obtaining.

An estimation system according to another aspect of the present disclosure estimates a blood glucose spike frequency of a subject. The estimation system includes: a measurement device that measures advanced glycation end products of the subject; an estimation device that estimates, using a correlation between a measurement value of advanced glycation end products and a blood glucose spike frequency prepared in advance, a blood glucose spike frequency of the subject based on a measurement value of the advanced glycation end products of the subject measured by the measurement device; and a display device that displays viewing information based on the blood glucose spike frequency of the subject estimated by the estimation device.

A determination method according to another aspect of the present disclosure determines a blood glucose spike frequency of a subject by a computing device. The determination method includes, as processing to be performed by the computing device: obtaining a measurement value of advanced glycation end products of the subject; comparing the measurement value of advanced glycation end products of the subject obtained in the obtaining with a standard measurement value of advanced glycation end products of a healthy individual; and when the measurement value of advanced glycation end products of the subject is larger than the standard measurement value of advanced glycation end products, determining that a blood glucose spike frequency of the subject is higher than a blood glucose spike frequency of the healthy individual.

An estimation marker according to another aspect of the present disclosure includes advanced glycation end products for estimating a blood glucose spike frequency.

### Advantageous Effects of Invention

The present disclosure allows for estimation of a blood glucose spike frequency based on a measurement value of advanced glycation end products of a subject without performing continuous glucose monitoring of the subject, thereby allowing a user to recognize the blood glucose spike frequency while suppressing a burden on the subject.

### BRIEF DESCRIPTION OF DRAWINGS

FIG. 1 is a diagram showing an estimation system according to a first embodiment.

FIG. 2 is a diagram showing exemplary transitions of blood glucose levels with respect to elapsed time according to the first embodiment.

FIG. 3 is a diagram showing a correlation between an AGE score and a blood glucose spike frequency according to the first embodiment.

FIG. 4 is a diagram showing the AGE score with respect to the blood glucose spike frequency according to the first embodiment.

FIG. 5 is a diagram showing a configuration of an estimation device according to the first embodiment.

FIG. 6 is a diagram for illustrating a user identification information table stored in the estimation device according to the first embodiment.

FIG. 7 is a diagram for illustrating a viewing information table stored in the estimation device according to the first embodiment.

FIG. 8 is a diagram showing an exemplary display screen in a display device according to the first embodiment.

FIG. 9 is a diagram showing an exemplary display screen in the display device according to the first embodiment.

FIG. 10 is a flowchart of an estimation process performed by the estimation device according to the first embodiment.

FIG. 11 is a diagram showing a correlation between an AGE score and a blood glucose spike frequency according to a second embodiment.

FIG. 12 is a diagram showing a density map summarizing a blood glucose level for each time frame in a first subject according to the second embodiment.

FIG. 13 is a diagram showing a table summarizing the blood glucose level for each time frame in the first subject according to the second embodiment.

FIG. 14 is a diagram showing a density map summarizing a blood glucose level for each time frame in a second subject according to the second embodiment.

FIG. 15 is a diagram showing a table summarizing the blood glucose level for each time frame in the second subject according to the second embodiment.

FIG. 16 is a diagram showing a density map summarizing a blood glucose level for each time frame in a third subject according to the second embodiment.

FIG. 17 is a diagram showing a table summarizing the blood glucose level for each time frame in the third subject according to the second embodiment.

FIG. 18 is a flowchart of a determination process performed by the estimation device according to a third embodiment.

### DESCRIPTION OF EMBODIMENTS

The present embodiment will be described in detail with reference to the drawings. The same or corresponding parts in the drawings are denoted by the same reference characters and description thereof will not be repeated in principle.

### First Embodiment

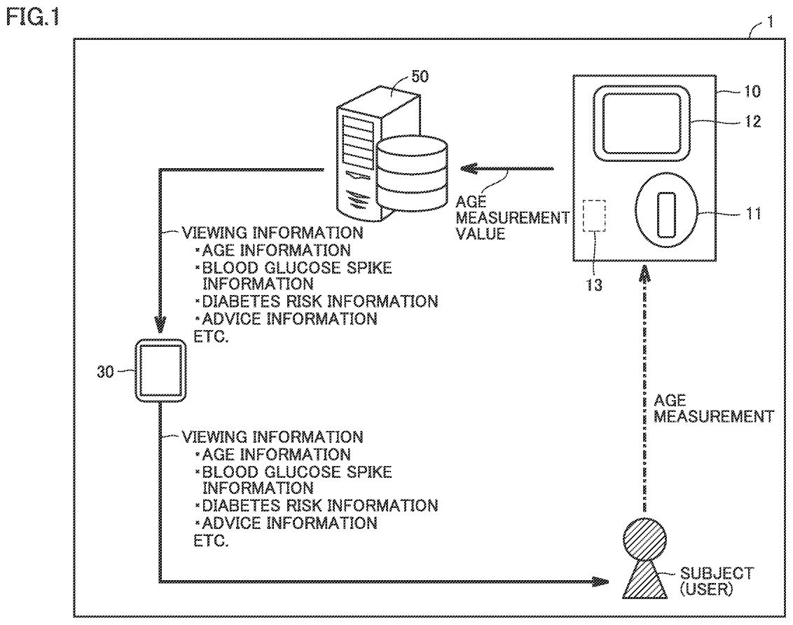

An estimation system 1 and an estimation device 50 according to a first embodiment will be described with reference to FIGS. 1 to 10.

### [Configuration of Estimation System]

FIG. 1 is a diagram showing estimation system 1 according to the first embodiment. As shown in FIG. 1, estimation system 1 includes an AGE measurement device 10, a display device 30, and an estimation device 50.

AGE measurement device 10 is a device for measuring AGEs of a subject. The subject includes an individual suspected to have developed a lifestyle-related disease such as diabetes or an age-related disease, an individual who has developed a lifestyle-related disease or an age-related disease, an elderly individual using a nursing care facility, and the like. AGE measurement device 10 includes a measurement unit 11, a display 12, and a communication unit 13. AGE measurement device 10 may be integrated with or separate from display 12.

Measurement unit 11 measures the AGEs of the subject in a non-invasive manner. Some of a plurality of compounds included in the AGEs have the property of emitting fluorescence by being irradiated with specific light. Measurement unit 11 uses such property of the compounds to measure the AGEs of the subject.

When the subject touches a fingertip to measurement unit 11, measurement unit 11 applies light to the skin from a not-shown light source. Measurement unit 11 may be configured to apply light to the skin (e.g., an arm) other than the fingertip of the subject. The light applied by measurement unit 11 is, for example, excitation light having a peak in a wavelength range equal to or less than 410 nm. Measurement unit 11 receives fluorescence excited by the light applied to the skin at a not-shown light receiving element, and measures a degree of accumulation of the AGEs based on the intensity of the received fluorescence. Display 12 displays a measurement result of the AGEs obtained by measurement unit 11. The measurement result includes, for example, the intensity of the fluorescence received by measurement unit 11, and a value of the degree of accumulation of the AGEs converted into a score. The measurement result may include the intensity of the fluorescence received by measurement unit 11, and a corrected value obtained by correcting the value of the degree of accumulation of the AGEs converted into a score.

Communication unit 13 transmits and receives data (information) to and from estimation device 50 by wired communication or wireless communication. Communication unit 13 may be a component capable of communicating with estimation device 50 such as a network adapter, and may be incorporated in AGE measurement device 10. Communication unit 13 may be an information terminal capable of communicating with estimation device 50 through a network, such as a desktop personal computer (PC), a laptop PC, a smartphone, a smart watch, a wearable device, and a tablet PC, and may be separate from AGE measurement device 10.

AGE measurement device 10 is placed in various types of facilities such as a pharmacy, a medical institution, a nursing care facility, and a gym. AGE measurement device 10 may be managed by a supporter who supports the subject. When the subject measures the AGEs using AGE measurement device 10, an AGE measurement value is transmitted from AGE measurement device 10 to estimation device 50.

Since the AGE measurement value generally changes in several weeks although there are individual differences, the subject measures the AGEs at a frequency of, for example, once in two weeks.

Display device 30 is owned or used by a user. Display device 30 is an information terminal capable of communicating with estimation device 50 through a network, such as a desktop PC, a laptop PC, a smartphone, a smart watch, a wearable device, and a tablet PC. By directly or indirectly accessing estimation device 50 using display device 30, the user can obtain various types of information such as advice information which will be described later stored in estimation device 50.

The user is a person who uses service provided by estimation system 1 (hereinafter also referred to as “information providing service”). Specifically, the user may be a subject, or a supporter of the subject. The user may be a family or a relative of the subject, or a related person (e.g., an acquaintance) relevant to the subject, who is authorized by the subject or the supporter to view the measurement result about the subject.

The supporter is a person who supports the subject, and includes: a staff member at a nursing care facility; a counselor at a nursing care facility; a doctor at a hospital, a clinic, or a corporate medical clinic; a nurse at a hospital, a clinic, or a corporate medical clinic; an instructor or a nutrition adviser at a fitness gym; and a pharmacist at a pharmacy.

Estimation device 50 is managed by a service provider that provides the information providing service. The service provider may be a manufacturer of AGE measurement device 10 that rents AGE measurement device 10 to the user such as the subject or the supporter. Estimation device 50 functions as a cloud computer, thereby communicating with each of AGE measurement device 10 and display device 30.

In estimation system 1 configured as described above, when the subject measures the AGEs using AGE measurement device 10, AGE measurement device 10 outputs the AGE measurement value to estimation device 50. When estimation device 50 obtains the AGE measurement value from AGE measurement device 10, estimation device 50 stores the obtained AGE measurement value together with AGE measurement values of the subject obtained in the past. Thus, the user of estimation system 1 can accumulate and observe the AGE measurement values of the subject, thereby preventing the development of lifestyle-related diseases or age-related diseases based on a change in the AGE measurement values.

As a method of checking to see if lifestyle habits, in particular dietary habits, are disordered, intermittently scanned CGM (is-CGM) of continuously monitoring a blood glucose level over a certain period of time (e.g., two weeks) is known. By performing the continuous glucose monitoring, a subject can check whether or not blood glucose spikes involving sharp rises and sharp drops in blood glucose level have occurred, and if so, a frequency of the blood glucose spikes (blood glucose spike frequency), and is motivated to review his/her lifestyle habits. In the case where the continuous glucose monitoring is performed, however, it is important to keep the continuous glucose monitoring to a necessary minimum because the subject goes about his/her daily life with a measuring needle inserted under the skin and the like for the certain period of time. The continuous glucose monitoring is also costly. Therefore, the continuous glucose monitoring is more burdensome to the subject and tends to be avoided by the subject, as compared to measurement of AGEs, which only needs to be done at a frequency of once a week or once in several weeks.

In estimation system 1 according to the first embodiment, therefore, estimation device 50 is configured to estimate the blood glucose spike frequency based on the AGE measurement value of the subject. Furthermore, estimation device 50 is configured to output, based on an estimation result of the blood glucose spike frequency, at least one of: blood glucose spike information about the estimation result of the blood glucose spike frequency; diabetes risk information indicative of a risk of diabetes (hereinafter, a risk associated with the development of a diabetic complication is also referred to as a “diabetes risk” for convenience, and information indicative of the diabetes risk is referred to as the “diabetes risk information”); and advice information indicative of advice on the life of the subject, as viewing information that can be viewed by the user such as the subject, the supporter, and a viewer.

Specifically, estimation device 50 estimates the blood glucose spike frequency of the subject based on the AGE measurement value obtained from AGE measurement device 10, and stores an estimation result of the blood glucose spike frequency together with estimation results of the blood glucose spike frequencies of the subject calculated in the past.

Estimation device 50 generates the blood glucose spike information based on the estimation result of the blood glucose spike frequency, and outputs the blood glucose spike information as the viewing information to display device 30. The blood glucose spike information includes at least one of: an estimation result of the blood glucose spike frequency of the subject at present or in the past; a value of the estimation result of the blood glucose spike frequency converted into a score; and a result of graded evaluation and ranking of the estimation result of the blood glucose spike frequency.

Estimation device 50 generates the diabetes risk information indicative of a risk of diabetes of the subject based on the estimation result of the blood glucose spike frequency, and outputs the diabetes risk information as the viewing information to display device 30. The diabetes risk information includes information indicative of a risk of diabetes (e.g., low risk, intermediate risk, high risk) of the subject at present or in the past.

Estimation device 50 generates the advice information indicative of advice on the life of the subject based on the estimation result of the blood glucose spike frequency, and stores the advice information as the viewing information. The advice information includes advice on at least one of eating habits, exercise habits, sleep habits, and mental health of the subject.

Estimation device 50 may generate the viewing information based on other types of information about the subject. The other types of information include, for example, data on a skeletal muscle mass index (hereinafter also referred to as “SMI”), inflammation, blood pressure, diet, exercise, vegetable intake, sleep, bone density, and the like. Estimation device 50 may analyze a health condition of the subject based on the other types of information described above, and include analysis information indicative of an analysis result of the health condition in the viewing information.

When the user requests the viewing information using display device 30, estimation device 50 outputs the viewing information to display device 30 in response to the request from display device 30. Display device 30 displays the viewing information obtained from estimation device 50.

As a result, the subject does not need to perform the continuous glucose monitoring, and the user can recognize the blood glucose spike frequency using display device 30 while suppressing the burden on the subject. Furthermore, the user can obtain, using display device 30, the risk of diabetes of the subject and the advice on the life of the subject generated based on the estimation result of the blood glucose spike frequency.

### [Correlation Between AGEs and Blood Glucose Spike Frequency]

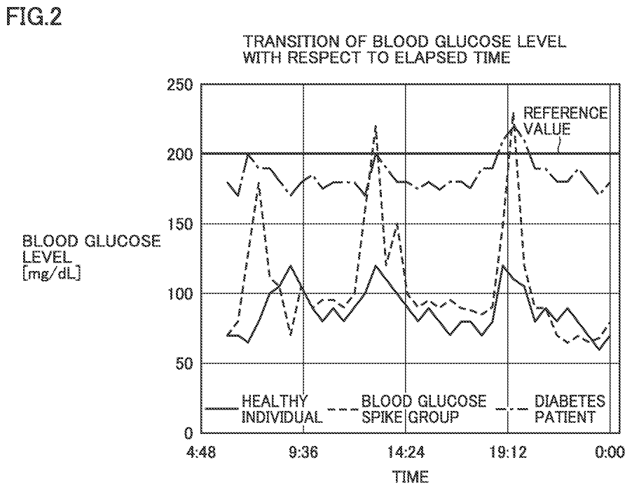

A correlation between AGEs and a blood glucose spike frequency will be described with reference to FIGS. 2 to 4. FIG. 2 is a diagram showing exemplary transitions of blood glucose levels with respect to elapsed time according to the first embodiment. FIG. 2 shows a graph representing variations in blood glucose level, with the horizontal axis representing time and the vertical axis representing the blood glucose level. Generally, a blood glucose level not exceeding 126 mg/dL is regarded as normal, whereas a blood glucose level exceeding 200 mg/dL at any given time is regarded as a basis for diagnosis of diabetes. As shown in FIG. 2, in blood glucose level data including the occurrence of blood glucose spikes, the blood glucose level varies rapidly, such as sharply rising to the same level as that of a diabetes patient and exceeding 200 mg/dL followed by a sharp drop shortly thereafter in a postprandial state, while being similar to that of a healthy individual in a fasting state.

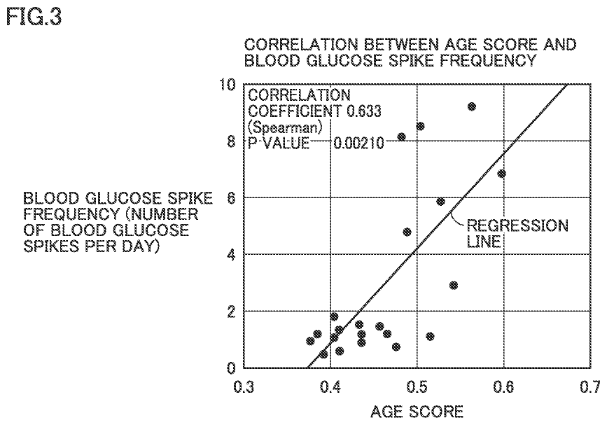

Although the occurrence of the blood glucose spikes shown in FIG. 2 can be found by performing the continuous glucose monitoring, ability to estimate the occurrence based on the AGE measurement value of the subject without performing the continuous glucose monitoring can suppress the burden on the subject. FIG. 3 is a diagram showing a correlation between an AGE score and a blood glucose spike frequency according to the first embodiment.

The correlation shown in FIG. 3 was created based on an AGE score and a blood glucose spike frequency of each subject, using a plurality of subjects who were healthy individuals as objects to be measured. Specifically, each subject first measures AGEs, and then measures a blood glucose level at predetermined intervals by the continuous glucose monitoring, regardless of whether the subject is postprandial, fasting, or sleeping, over a predetermined period of time after measuring the AGEs. In this example, each subject first measured the AGEs, and then measured the blood glucose level at one minute intervals by the continuous glucose monitoring, regardless of whether the subject was postprandial, fasting, or sleeping, for two weeks after measuring the AGEs. Since the AGEs change to a lesser extent than the blood glucose level and may generally change in several weeks, each subject only needs to measure the AGEs at least once in one to two weeks. If the AGEs are measured a plurality of times, a plurality of AGE measurement values obtained may be simply averaged. In this example, each subject measured the AGEs only once in two weeks.

A designer of estimation system 1 and estimation device 50 collects the AGEs and the blood glucose levels of each subject measured in two weeks, and calculates an AGE score and a blood glucose spike frequency of each subject. Specifically, the designer converts, for each subject, the obtained AGE measurement value into an AGE score between 0 and 1.0. Furthermore, the designer calculates, for each subject, the number of data exceeding 200 mg/dL of a plurality of blood glucose level data obtained at one minute intervals for two weeks, and divides the calculated number of data exceeding 200 mg/dL by 14 (that is, the number of days of two weeks), thereby calculating the number of blood glucose spikes per day (blood glucose spike frequency). The measurement period of the blood glucose level is not limited to two weeks, but may be several days or one month.

As shown in FIG. 3, by plotting a dot at a position corresponding to the AGE score and the blood glucose spike frequency of each subject in a graph with the horizontal axis representing the AGE score and the vertical axis representing the blood glucose spike frequency, the designer can create a graph representing the correlation between the AGE score and the blood glucose spike frequency. Each dot shown in FIG. 3 indicates the AGE score and the blood glucose spike frequency of each subject.

As shown in FIG. 3, as the AGE score is larger, the blood glucose spike frequency is higher, and as the AGE score is smaller, the blood glucose spike frequency is lower. For example, there is a correlation between the AGE score and the blood glucose spike frequency with a correlation coefficient of 0.633. Here, the P value is the probability of observing, under the null hypothesis, a statistic that contradicts the hypothesis more extremely than a statistic calculated from actual data. In the example of FIG. 3, the P value is 0.0021, which is lower than 0.05, indicating that the correlation between the AGE score and the blood glucose spike frequency has some degree of reliability, which is unlikely to be coincidental. Thus, it can be said that there is a relatively strong correlation between the AGE score and the blood glucose spike frequency.

As shown in FIG. 3, a regression line can be drawn for the correlation between the AGE score and the blood glucose spike frequency, and estimation device 50 can estimate the blood glucose spike frequency corresponding to the AGE score using such a regression line. Specifically, estimation device 50 can predict the blood glucose spike frequency of the subject by substituting the AGE measurement value of the subject into an equation of the regression line described above.

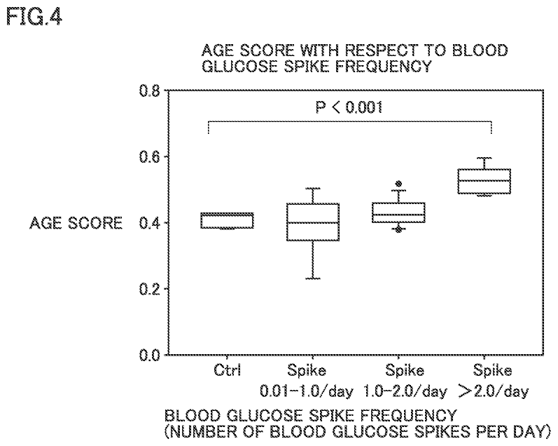

FIG. 4 is a diagram showing the AGE score with respect to the blood glucose spike frequency according to the first embodiment. FIG. 4 shows a graph with the horizontal axis representing the blood glucose spike frequency and the vertical axis representing the AGE score. The graph shown in FIG. 4 represents a quartile range of the AGE score for each of the cases where the blood glucose spike frequency per day is 0 times, 0.01 to 1 times, 1 to 2 times, and higher than 2 times. As shown in FIG. 4, the AGE score does not change significantly when the blood glucose spike frequency is equal to or lower than 2 times, whereas the AGE score increases significantly when the blood glucose spike frequency is higher than 2 times. That is, when the blood glucose spike frequency is higher than 2 times, it is highly likely that lifestyle-related diseases such as diabetes develop.

Thus, there is a correlation between the AGEs and the blood glucose spike frequency, and estimation device 50 is configured to estimate, using data indicative of such a correlation (hereinafter also referred to as “correlation data”), the blood glucose spike frequency based on the AGE measurement value of the subject. Specifically, estimation device 50 can convert the AGE measurement value of the subject obtained from AGE measurement device 10 regardless of whether the subject is postprandial, fasting, or sleeping, into the AGE score, and estimate, using the correlation data shown in FIG. 3, the blood glucose spike frequency of the subject based on the converted AGE score. The correlation data is not limited to the correlation between the AGE score and the blood glucose spike frequency shown in FIG. 3, but may be data indicative of a correlation between the AGE measurement value and the blood glucose spike frequency. In this case, estimation device 50 may use the AGE measurement value obtained from AGE measurement device 10 without any change, and estimate the blood glucose spike frequency based on the AGE measurement value.

### [Configuration of Estimation Device]

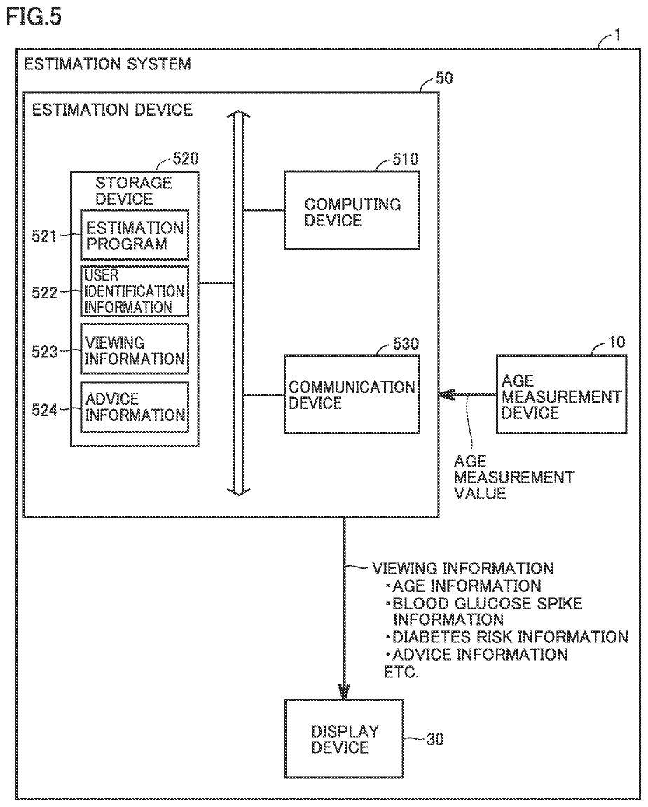

A configuration of estimation device 50 will be described with reference to FIGS. 5 to 7. FIG. 5 is a diagram showing the configuration of estimation device 50 according to the first embodiment. As shown in FIG. 5, estimation device 50 includes a computing device 510, a storage device 520, and a communication device 530.

Computing device 510 is a computer (computing entity) that performs various types of processing in accordance with various programs. Computing device 510 is implemented by a computer such as a processor. The processor is implemented by, for example, a microcontroller, a central processing unit (CPU), or a micro-processing unit (MPU). Although the processor performs functions to perform various types of processing by executing a program, some or all of these functions may be performed by dedicated hardware circuitry such as an application specific integrated circuit (ASIC), a graphics processing unit (GPU), or a field-programmable gate array (FPGA). The “processor” is not limited to a processor in a narrow sense that performs processing in accordance with a stored program architecture like the CPU or the MPU, but may encompass hard-wired circuitry such as the ASIC, the GPU, or the FPGA. Thus, the processor can also be read as processing circuitry, in which processing is predefined by a computer readable code and/or hard-wired circuitry. The processor may be implemented by a single chip or a plurality of chips. Furthermore, the processor and related processing circuitry may be implemented by a plurality of computers interconnected in a wired or wireless manner over a local area network or a wireless network. The processor and the related processing circuitry may be implemented by a cloud computer that performs remote computation based on input data and outputs a computation result to another device located at a remote site.

Furthermore, computing device 510 may include a storage unit for storing a program code or a work memory in execution of various programs by the processor. The storage unit may be one or more non-transitory computer readable media. The storage unit may include a volatile memory such as a dynamic random access memory (DRAM) and a static random access memory (SRAM), or a non-volatile memory such as a read only memory (ROM) and a flash memory. The storage unit may be one or more computer readable storage media. Examples of the storage unit include a storage device such as a hard disk drive (HDD) and a solid state drive (SSD).

Storage device 520 is one or more computer readable storage media, and includes a hard disk drive (HDD), a solid state drive (SSD) and the like. Storage device 520 stores various types of programs and data such as an estimation program 521 executed by computing device 510, user identification information 522 referenced to by computing device 510, viewing information 523 that can be viewed by the user using display device 30, and advice information 524 prepared in advance.

Computing device 510 may include a not-shown media reader. Computing device 510 may receive, through the media reader, a removable disk which is one or more computer readable storage media, and obtain the various types of programs and data such as estimation program 521, user identification information 522, and advice information 524 from the removable disk.

Estimation program 521 specifies various instructions to be executed by computing device 510 to estimate, using the correlation data between the AGE score and the blood glucose spike frequency shown in FIG. 3, the blood glucose spike frequency based on the AGE measurement value of the subject. The correlation data indicative of the correlation between the AGE score and the blood glucose spike frequency is stored in storage device 520 in advance.

User identification information 522 includes information about the user, such as a user ID, a password, and a user name. Estimation device 50 can identify the user using user identification information 522.

Viewing information 523 includes: subject information including information about the subject; AGE information including information about the AGE measurement value of the subject obtained from AGE measurement device 10; blood glucose spike information including information about the blood glucose spike frequency of the subject estimated based on the AGE measurement value; diabetes risk information including information about a diabetes risk of the subject generated based on the estimation result of the blood glucose spike frequency; and advice information generated based on the estimation result of the blood glucose spike frequency.

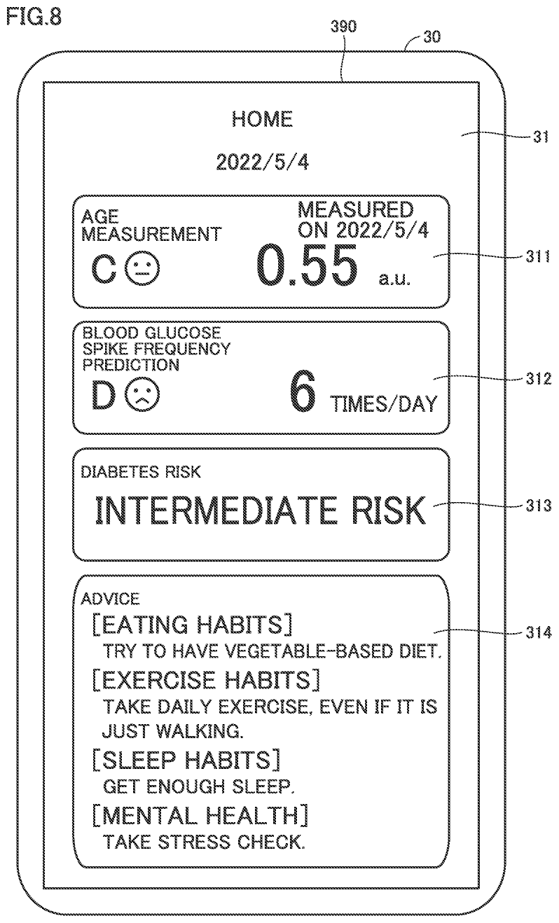

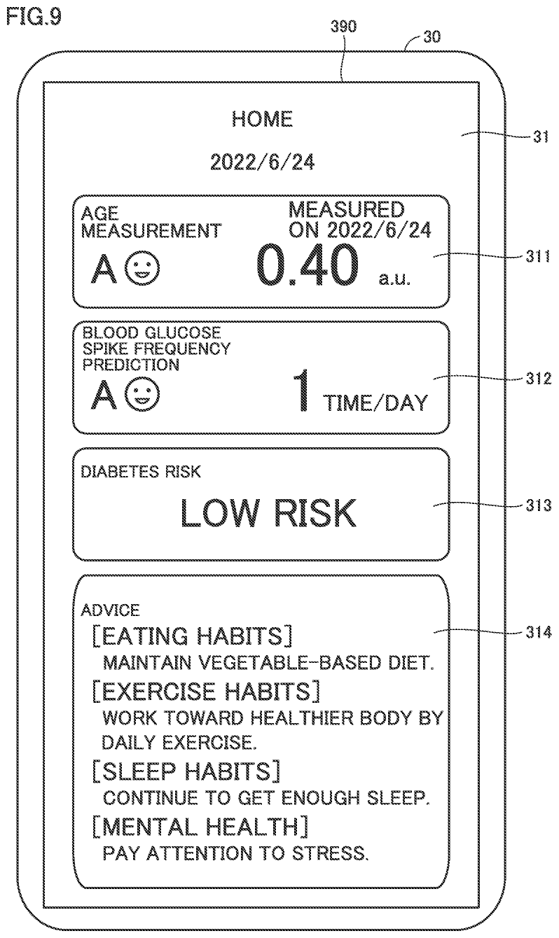

Advice information 524 includes a plurality of types of advice on the life of the subject, and is stored in storage device 520 such that they can be selected depending on the estimation result of the blood glucose spike frequency. Computing device 510 selects at least one of the plurality of types of advice included in advice information 524 based on the estimation result of the blood glucose spike frequency, and includes the selected advice in viewing information 523. The advice on the life of the subject includes, for example, advice on eating habits, exercise habits, sleep habits, mental health, and an inflammatory state caused by injury or disease of the subject, as shown in an image 314 shown in FIGS. 8 and 9 which will be described later.

Communication device 530 receives the AGE measurement value from AGE measurement device 10 by wired communication or wireless communication. Furthermore, communication device 530 transmits the viewing information to display device 30 by wired communication or wireless communication.

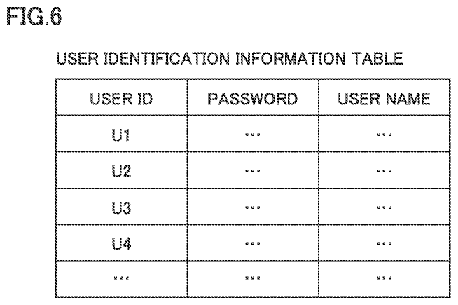

FIG. 6 is a diagram for illustrating a user identification information table stored in estimation device 50 according to the first embodiment. Estimation device 50 stores user identification information 522 using the user identification information table shown in FIG. 6.

As shown in FIG. 6, the user identification information table stores, as user identification information 522, various types of information about the user, such as a user ID, a password, and a user name. Each user who uses the information providing service is identified by user identification information 522. For example, “U1” is assigned to a first user as the user ID, and “U2” is assigned to a second user as the user ID.

Of user identification information 522, the user ID, the password, and the user name are input from display device 30 by each user. Display device 30 outputs, to estimation device 50, user identification information 522 input to display device 30. Estimation device 50 stores user identification information 522 obtained from display device 30 in the user identification information table, thereby storing user identification information 522 in storage device 520.

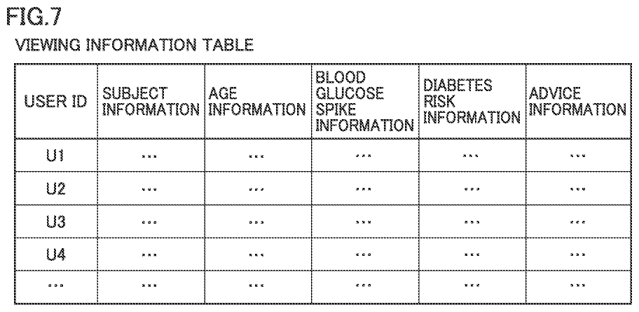

FIG. 7 is a diagram for illustrating a viewing information table stored in estimation device 50 according to the first embodiment. Estimation device 50 stores viewing information 523 using the viewing information table shown in FIG. 7.

As shown in FIG. 7, the viewing information table stores various types of information that can be viewed by the user, such as the subject information, the AGE information, the blood glucose spike information, the diabetes risk information, and the advice information, in association with the user ID.

The AGE information includes at least one of: an AGE measurement value at present or in the past obtained from AGE measurement device 10; a value of the AGE measurement value converted into an AGE score; and a result of ranking the AGE score. The AGE score includes, for example, a value of the AGE measurement value converted into each score between 0 and 1.0, as shown in FIGS. 3 and 4. The rank of the AGE score includes a result of ranking the AGE score by five-grade evaluation from A to E, as shown in FIGS. 8 and 9 which will be described later.

The blood glucose spike information includes at least one of: an estimation result of the blood glucose spike frequency at present or in the past estimated based on the AGE measurement value; a value of the estimation result of the blood glucose spike frequency converted into a score; and a result of ranking the blood glucose spike frequency. For example, the score of the blood glucose spike frequency includes a value of the estimation result of the blood glucose spike frequency converted into each score between 0 and 10, as shown in FIGS. 8 and 9 which will be described later. The rank of the blood glucose spike frequency includes a result of ranking the blood glucose spike frequency by five-grade evaluation from A to E, as shown in FIGS. 8 and 9 which will be described later.

The diabetes risk information includes information about a diabetes risk generated based on the estimation result of the blood glucose spike frequency. Estimation device 50 stores diabetes risks corresponding to blood glucose spike frequencies in advance, and upon estimating a blood glucose spike frequency based on the AGE measurement value, obtains a diabetes risk corresponding to the estimated blood glucose spike frequency and stores the diabetes risk in the viewing information table (viewing information 523). The information about a diabetes risk includes, for example, information for notifying the user of the diabetes risk of the subject, as shown in an image 313 shown in FIGS. 8 and 9 which will be described later.

### [Display Examples of Viewing Information]

Display examples of the viewing information will be described with reference to FIGS. 8 and 9. FIGS. 8 and 9 are diagrams showing exemplary display screens in display device 30 according to the first embodiment.

When the user executes an application program for using the information providing service using display device 30, display device 30 causes a display 390 to display a not-shown login screen. When the user inputs a user ID and a password on the login screen, display device 30 outputs the user ID and the password to estimation device 50. When estimation device 50 authenticates the user based on the user ID and the password, display device 30 causes display 390 to display a home screen 31 shown in FIG. 8.

Home screen 31 includes an image 311 for viewing the AGE information, an image 312 for viewing the blood glucose spike information, image 313 for viewing the diabetes risk information, and image 314 for viewing the advice information.

Image 311 shows, for example, an AGE score corresponding to the most recently measured AGE measurement value, and a result of ranking the AGE score. Here, the rank of the AGEs is a value indicative of an evaluation of the AGE score calculated by estimation device 50 based on a plurality of graded reference values. For example, as the AGE measurement value is smaller, the evaluation rank of the AGEs is closer to “A”, with the evaluation rank “A” of the AGEs indicating the highest evaluation of the AGE measurement value, and as the AGE measurement value is larger, the evaluation rank of the AGEs is closer to “E”, with the evaluation rank “E” of the AGEs indicating the lowest evaluation of the AGE measurement value. In the example of FIG. 8, image 311 shows “0.55” as the AGE score before improvement measured on May 5, 2022, and shows “C” as the rank of the AGEs. In the example of FIG. 9, image 311 shows “0.40” as the AGE score after improvement measured on Jun. 24, 2022, and shows “A” as the rank of the AGEs.

Although not shown, when the user selects (e.g., touches) image 311, display device 30 displays the most recently measured AGE measurement value, a time-series change in the AGE measurement values in the past, a comment on the AGE measurement value, and the like. As a result, the user can view the AGE information of the subject using display device 30.

Image 312 shows, for example, an estimation result of the blood glucose spike frequency estimated based on the most recently measured AGE measurement value, and a result of ranking the blood glucose spike frequency. Here, the rank of the blood glucose spike frequency is a value indicative of an evaluation of the blood glucose spike frequency calculated by estimation device 50 based on a plurality of graded reference values. For example, as the blood glucose spike frequency is lower, the evaluation rank of the blood glucose spike frequency is closer to “A”, with the evaluation rank “A” of the blood glucose spike frequency indicating the highest evaluation of the blood glucose spike frequency, and as the blood glucose spike frequency is higher, the evaluation rank of the blood glucose spike frequency is closer to “E”, with the evaluation rank “E” of the blood glucose spike frequency indicating the lowest evaluation of the blood glucose spike frequency. In the example of FIG. 8, image 312 shows “6 times/day” as the blood glucose spike frequency estimated based on the AGE score before improvement measured on May 4, 2022, and shows “D” as the rank of the blood glucose spike frequency. In the example of FIG. 9, image 312 shows “1 time/day” as the blood glucose spike frequency estimated based on the AGE score after improvement measured on Jun. 24, 2022, and shows “A” as the rank of the blood glucose spike frequency.

Thus, the blood glucose spike information shown in image 312 changes depending on the AGE information shown in image 311. Specifically, as the AGE score is larger, the blood glucose spike frequency is higher, and as the AGE score is smaller, the blood glucose spike frequency is lower. In addition, as the rank of the AGE score is worse, the rank of the blood glucose spike frequency is worse, and as the rank of the AGE score is better, the rank of the blood glucose spike frequency is better.

Although not shown, when the user selects (e.g., touches) image 312, display device 30 displays an estimation result of the blood glucose spike frequency estimated based on the most recently measured AGE measurement value, a time-series change in the blood glucose spike frequencies estimated based on the AGE measurement values in the past, a comment on the blood glucose spike frequency, and the like. As a result, the user can view the blood glucose spike information of the subject using display device 30.

Image 313 shows, for example, a diabetes risk corresponding to the estimated blood glucose spike frequency. In the example of FIG. 8, image 313 shows an “intermediate risk” as the diabetes risk corresponding to the blood glucose spike frequency estimated based on the AGE score before improvement measured on May 4, 2022. In the example of FIG. 9, image 313 shows a “low risk” as the diabetes risk corresponding to the blood glucose spike frequency estimated based on the AGE score after improvement measured on Jun. 24, 2022.

Thus, the diabetes risk information shown in image 313 changes depending on the blood glucose spike information shown in image 312. Specifically, as the blood glucose spike frequency is higher, the diabetes risk is higher, and as the blood glucose spike frequency is lower, the diabetes risk is lower. In addition, as the rank of the blood glucose spike frequency is worse, the diabetes risk is higher, and as the rank of the blood glucose spike frequency is better, the diabetes risk is lower.

Image 314 shows, as advice corresponding to the estimated blood glucose spike frequency, for example, advice on eating habits, exercise habits, sleep habits, and mental health. In the example of FIG. 8, image 314 shows, as advice corresponding to the blood glucose spike frequency estimated based on the AGE score before improvement measured on May 4, 2022, recommendations to try to have a vegetable-based diet, to exercise such as walking, to get enough sleep, and to take a stress check. In the example of FIG. 9, image 314 shows, as advice corresponding to the blood glucose spike frequency estimated based on the AGE score after improvement measured on Jun. 24, 2022, recommendations to maintain the vegetable-based diet, to work toward a healthier body by daily exercise, to continue to get enough sleep, and to pay attention to stress.

Thus, the advice information shown in image 314 changes depending on the blood glucose spike information shown in image 312. Specifically, as the blood glucose spike frequency is higher, advice that encourages taking better care of health is shown, such as significantly improving the eating habits, exercise habits, sleep habits, and mental health. As the blood glucose spike frequency is lower, advice that acknowledges the good daily habits of the subject is shown, such as maintaining the improved eating habits, exercise habits, sleep habits, and mental health.

Thus, the user can view the AGE information, the blood glucose spike information, the diabetes risk information, the advice information and the like, and can further view times-series changes in these types of information, using display device 30.

### [Process of Estimation Device]

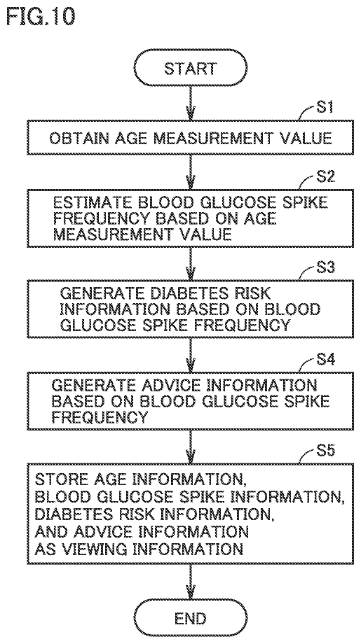

A process of estimation device 50 will be described with reference to FIG. 10. FIG. 10 is a flowchart of an estimation process performed by estimation device 50 according to the first embodiment. Process steps (hereinafter abbreviated as “S”) shown in FIG. 10 are implemented by computing device 510 executing estimation program 521.

As shown in FIG. 10, estimation device 50 obtains an AGE measurement value from AGE measurement device 10 (S1). Estimation device 50 estimates a blood glucose spike frequency based on the obtained AGE measurement value (S2).

Estimation device 50 generates diabetes risk information based on an estimation result of the blood glucose spike frequency (S3). Estimation device 50 generates advice information based on the estimation result of the blood glucose spike frequency (S4).

Estimation device 50 stores the AGE information, the blood glucose spike information, the diabetes risk information, and the advice information in storage device 520 as viewing information 523 (S5). As a result, estimation device 50 outputs the AGE information, the blood glucose spike information, the diabetes risk information, and the advice information stored as viewing information 523 to display device 30 in response to a request from display device 30, thereby allowing the user to view these types of information.

As described above, in estimation device 50 according to the first embodiment, the blood glucose spike frequency can be estimated based on the AGE measurement value measured by AGE measurement device 10, and the estimated blood glucose spike frequency can be provided to the user. As a result, the subject does not need to perform the continuous glucose monitoring, and the user can recognize the blood glucose spike frequency using display device 30 while suppressing the burden on the subject. Furthermore, the user can obtain, using display device 30, the risk of diabetes of the subject and the advice on the life of the subject generated based on the estimation result of the blood glucose spike frequency.

### Second Embodiment

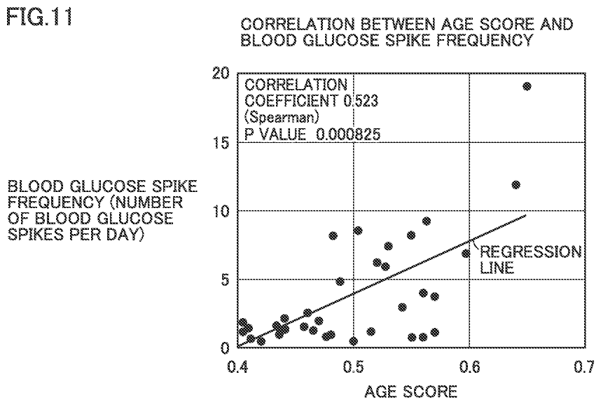

Estimation system 1 and estimation device 50 according to a second embodiment will be described with reference to FIGS. 11 to 17. Estimation system 1 and estimation device 50 according to the second embodiment will be described below in terms of differences relative to estimation system 1 and estimation device 50 according to the first embodiment, and the same portions as those of estimation system 1 and estimation device 50 according to the first embodiment may not be described.

FIG. 11 is a diagram showing a correlation between an AGE score and a blood glucose spike frequency according to the second embodiment. Similarly to the correlation shown in FIG. 3 according to the first embodiment, the correlation shown in FIG. 11 according to the second embodiment was created based on an AGE score and a blood glucose spike frequency of each subject, using a plurality of subjects as objects to be measured. Specifically, each subject first measures AGEs, and then measures a blood glucose level at one minute intervals, regardless of whether the subject is postprandial, fasting, or sleeping, for two weeks after measuring the AGEs. The designer of estimation system 1 and estimation device 50 converts, for each subject, the obtained AGE measurement value into an AGE score between 0 and 10.0. Furthermore, the designer calculates, for each subject, the number of data exceeding 200 mg/dL of a plurality of blood glucose level data obtained at one minute intervals for two weeks, and divides the calculated number of data exceeding 200 mg/dL by 14 (that is, the number of days of two weeks), thereby calculating the number of blood glucose spikes per day (blood glucose spike frequency). The measurement period of the blood glucose level is not limited to two weeks, and the blood glucose level can be evaluated with a measurement period of several days or one month.

As shown in FIG. 11, by plotting a dot at a position corresponding to the AGE score and the blood glucose spike frequency of each subject in a graph with the horizontal axis representing the AGE score and the vertical axis representing the blood glucose spike frequency, the designer can create a graph representing the correlation between the AGE score and the blood glucose spike frequency. Each dot shown in FIG. 11 indicates the AGE score and the blood glucose spike frequency of each subject.

Similarly to the graph shown in FIG. 3 according to the first embodiment, in the graph shown in FIG. 11 as well, as the AGE score is larger, the blood glucose spike frequency is higher, and as the AGE score is smaller, the blood glucose spike frequency is lower. For example, there is a correlation between the AGE score and the blood glucose spike frequency with a correlation coefficient of 0.523. Furthermore, in the example of FIG. 11, the P value is 0.000825, which is lower than 0.05, indicating that the correlation between the AGE score and the blood glucose spike frequency has some degree of reliability, which is unlikely to be coincidental.

As shown in FIG. 11, a regression line can be drawn for the correlation between the AGE score and the blood glucose spike frequency, and estimation device 50 can estimate the blood glucose spike frequency corresponding to the AGE score using such a regression line.

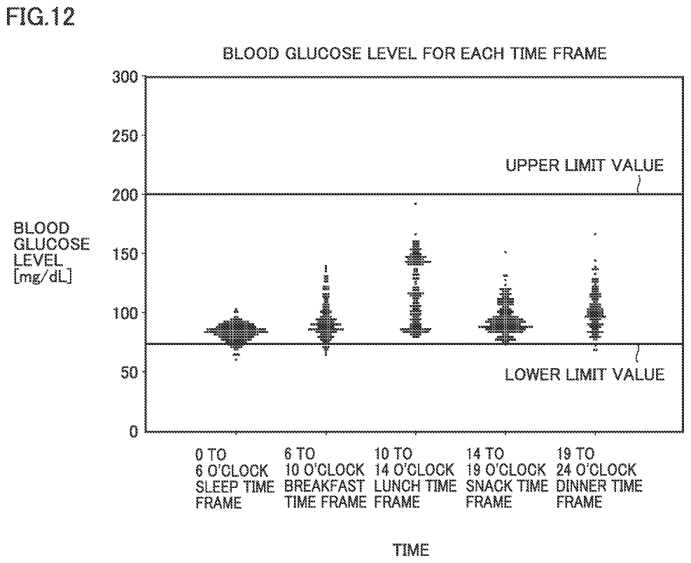

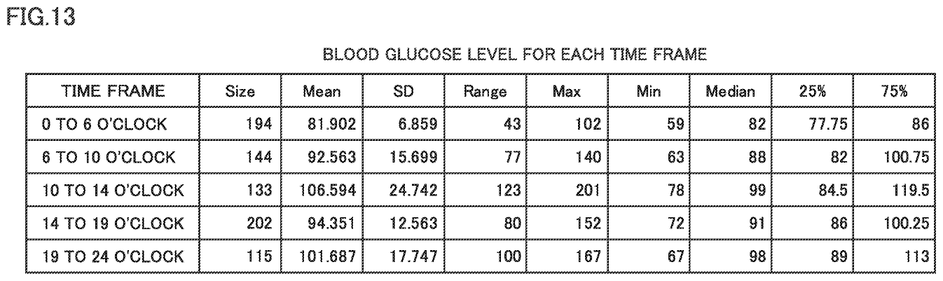

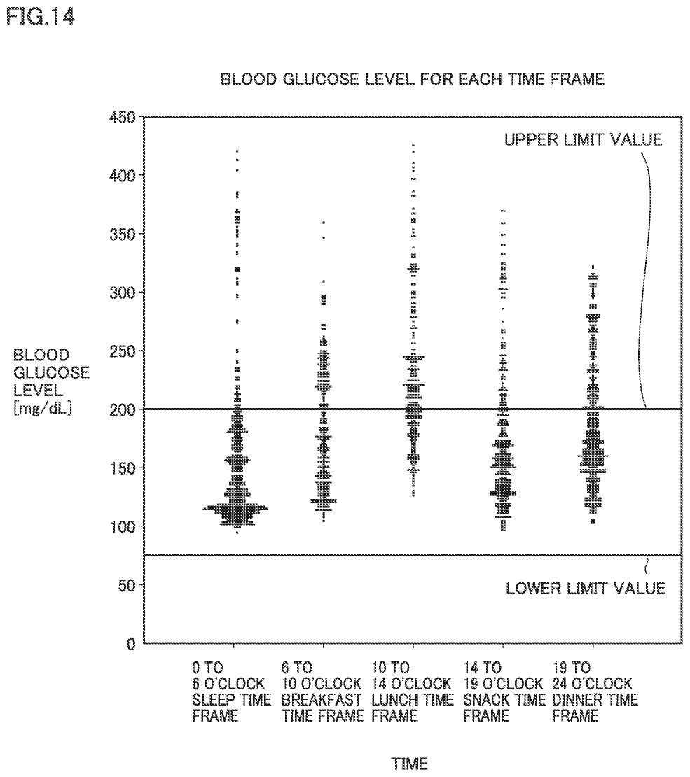

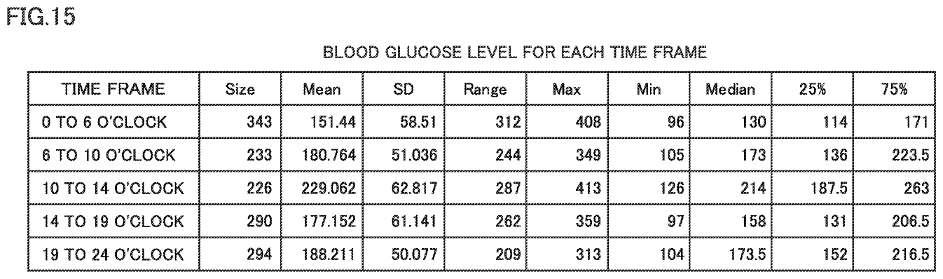

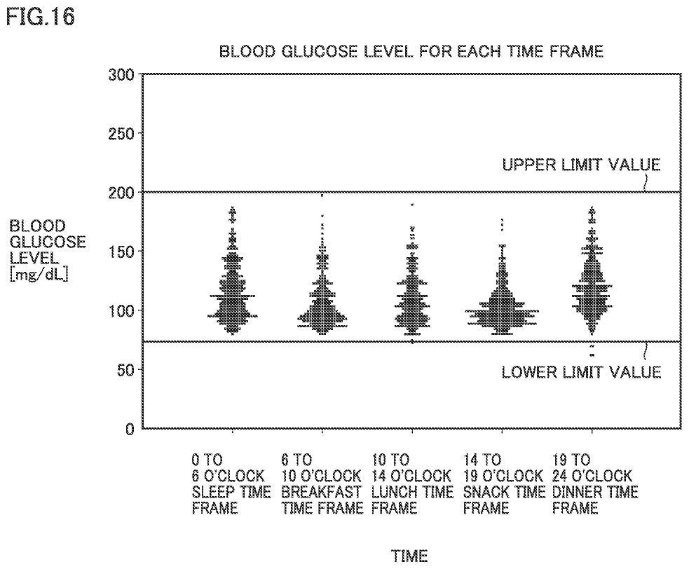

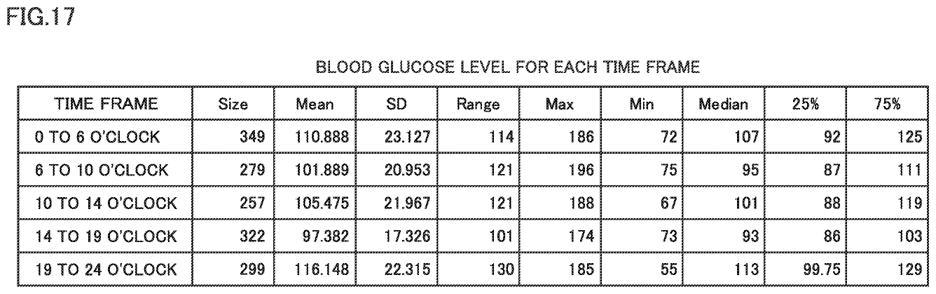

Here, estimation device 50 according to the second embodiment is configured to find potential diabetics suspected to develop diabetes using the correlation shown in FIG. 11. When creating the correlation shown in FIG. 11, therefore, subjects who are chronically in a hyperglycemic state and subjects in which blood glucose spikes do not occur are excluded from the objects to be measured, and only gray-zone subjects suspected of being potential diabetics are employed as the objects to be measured during the creation of the correlation. The subjects during the creation of the correlation according to the second embodiment will be described with reference to FIGS. 12 to 17. FIGS. 12 and 13 show a trend of a blood glucose level for a first subject, FIGS. 14 and 15 show a trend of a blood glucose level for a second subject, and FIGS. 16 and 17 show a trend of a blood glucose level for a third subject.

FIGS. 12, 14 and 16 are diagrams showing density maps summarizing the blood glucose levels for each time frame in the first subject, the second subject, and the third subject according to the second embodiment, respectively. FIGS. 12, 14 and 16 each show a graph with the horizontal axis representing a plurality of time frames and the vertical axis representing the blood glucose level, and a lower limit value of the blood glucose level corresponding to hypoglycemia and an upper limit value of the blood glucose level corresponding to hyperglycemia are set in the graph. For example, 70 mg/dL is employed as the lower limit value and 200 mg/dL is employed as the upper limit value. The plurality of time frames include a sleep time frame from 0 to 6 o'clock, a breakfast time frame from 6 to 10 o'clock, a lunch time frame from 10 to 14 o'clock, a snack time frame from 14 to 19 o'clock, and a dinner time frame from 19 to 24 o'clock. The designer can create the density maps shown in FIGS. 12, 14 and 16 by aggregating, for each time frame, a plurality of blood glucose level data obtained at one-minute intervals for two weeks for each of the first subject, the second subject, and the third subject.

FIGS. 13, 15 and 17 are diagrams showing tables summarizing the blood glucose levels for each time frame in the first subject, the second subject, and the third subject according to the second embodiment, respectively. FIGS. 13, 15 and 17 each show a table summarizing, for each time frame, the number of data (Size), a mean value (Mean), a standard deviation (SD), a difference between a minimum value and a maximum value (Range), the maximum value (Max), the minimum value (Min), a median value (Median), a first quartile (Q1, 25%), and a third quartile (Q3, 75%), of the blood glucose level.

As shown in FIG. 12, the blood glucose level of the first subject generally lies between the lower limit value (70 mg/dL) and the upper limit value (200 mg/dL) in all of the time frames. In addition, as shown in FIG. 13, the difference between the minimum value of the blood glucose level and the maximum value of the blood glucose level (Range) in the first subject is “123” at a maximum in the time frame from 10 to 14 o'clock, which is equal to or smaller than a predetermined value (e.g., 130) preset by the designer. It can thus be said that the first subject is not chronically in a hyperglycemic state. The predetermined value can be set as appropriate by the designer. Furthermore, as shown in FIGS. 12 and 13, the blood glucose level of the first subject exceeds the upper limit value (200 mg/dL) in the time frame from 10 to 14 o'clock. The blood glucose spike frequency of the first subject is 0.1 times per day. It can thus be said that blood glucose spikes can occur in the first subject. Therefore, the designer assumes that the first subject is suspected of being a potential diabetic, and employs the first subject as the object to be measured when creating the correlation shown in FIG. 11.

As shown in FIG. 14, the blood glucose level of the second subject chronically exceeds the upper limit value (200 mg/dL) in all of the time frames. In addition, as shown in FIG. 15, the difference between the minimum value of the blood glucose level and the maximum value of the blood glucose level (Range) in the second subject exceeds the predetermined value (e.g., 130) preset by the designer in all of the time frames. Furthermore, the blood glucose spike frequency of the second subject is 32.1 times per day. It can thus be said that the second subject is chronically in a hyperglycemic state. Therefore, the designer assumes that the second subject is a patient who is chronically in a hyperglycemic state, and excludes the second subject from the objects to be measured when creating the correlation shown in FIG. 11.

As shown in FIG. 16, the blood glucose level of the third subject lies between the lower limit value (70 mg/dL) and the upper limit value (200 mg/dL) in all of the time frames. In addition, as shown in FIG. 17, the difference between the minimum value of the blood glucose level and the maximum value of the blood glucose level (Range) in the third subject is “130” at a maximum in the time frame from 19 to 24 o'clock, which is equal to or smaller than the predetermined value (e.g., 130) preset by the designer. It can thus be said that the first subject is not chronically in a hyperglycemic state. As shown in FIGS. 16 and 17, however, the blood glucose level of the third subject does not exceed the upper limit value (200 mg/dL) in any of the time frames. The blood glucose spike frequency of the third subject is 0 times per day. It can thus be said that blood glucose spikes cannot occur in the third subject. Therefore, the designer assumes that the third subject is a subject in which blood glucose spikes do not occur, and excludes the third subject from the objects to be measured when creating the correlation shown in FIG. 11.

As described above, in a subject who is chronically in a hyperglycemic state (i.e., a subject already affected with diabetes), the difference between the minimum value and the maximum value of the blood glucose level (Range) tends to be large in all of the time frames. In view of this, the designer of estimation device 50 assumes that subjects whose difference between the minimum value and the maximum value of the blood glucose level (Range) in a particular time frame exceeds the predetermined value (e.g., 130) are subjects who are chronically in a hyperglycemic state, and excludes them from the objects when creating the correlation. To estimate whether or not the subject is a potential diabetic, it is desirable to create the correlation using measurement results of subjects who are potential diabetics. Therefore, when the correlation is created by excluding the subjects who are chronically in a hyperglycemic state from the objects for the creation of the correlation as described above, the accuracy of estimation of whether or not the subject is a potential diabetic is improved as compared to when the correlation is created without excluding the subjects who are chronically in a hyperglycemic state from the objects for the creation of the correlation. The reason for not distinguishing between subjects who are chronically in a hyperglycemic state and subjects who are not chronically in a hyperglycemic state (i.e., subjects not affected with diabetes) based on the magnitude of the blood glucose spike frequency is that, for example, blood glucose spikes may occur numerous times temporarily after eating even in the subjects who are not chronically in a hyperglycemic state, and simply distinguishing between the two based on the magnitude of the blood glucose spike frequency fails to identify subjects who are “chronically” in a hyperglycemic state.

Furthermore, of the subjects who are not chronically in a hyperglycemic state identified based on the comparison of the difference between the minimum value and the maximum value of the blood glucose level (Range) with the predetermined value, it is preferable to distinguish between subjects who are potential diabetics and subjects who are not potential diabetics based on the blood glucose spike frequency per day, and exclude the subjects who are not potential diabetics from the objects to be measured for the creation of the correlation. With such a configuration, only the subjects who are potential diabetics are employed as the objects to be measured for the creation of the correlation, which leads to further improved accuracy of estimation of whether or not the subject is a potential diabetic. Subjects who are potential diabetics and subjects who are not potential diabetics may be distinguished based on not only the blood glucose spike frequency per day, but also the blood glucose spike frequency for several days (a plurality of days). Furthermore, even subjects in which blood glucose spikes occurred a predetermined number of times may be included in the subjects who are not potential diabetics.

Thus, estimation device 50 according to the second embodiment is configured to estimate the blood glucose spike frequency of the subject based on the AGE measurement value obtained from AGE measurement device 10, using the correlation shown in FIG. 11 created using, as the objects to be measured, the subjects identified based on the comparison of the value relating to blood glucose level that differs between a subject affected with diabetes and a subject not affected with diabetes with the predetermined value. Specifically, estimation device 50 is configured to estimate the blood glucose spike frequency of the subject based on the AGE measurement value obtained from AGE measurement device 10, using the correlation shown in FIG. 11 created using, as the objects to be measured, the subjects suspected of being potential diabetics whose difference between the minimum value of the blood glucose level and the maximum value of the blood glucose level (Range) is equal to or smaller than the predetermined value (e.g., 130). As a result, estimation device 50 can estimate the blood glucose spike frequency of the subject using the correlation shown in FIG. 11 created using the subjects suspected of being potential diabetics as the objects, and can therefore estimate whether or not the subject is a potential diabetic based on the estimated blood glucose spike frequency.

The correlation between the AGE measurement value and the blood glucose spike frequency may be created using, as the objects to be measured, subjects whose difference between the minimum value of the blood glucose level and the maximum value of the blood glucose level (Range) is equal to or smaller than a predetermined value in all of the time frames, or may be created using, as the objects to be measured, subjects whose difference between the minimum value of the blood glucose level and the maximum value of the blood glucose level (Range) is equal to or smaller than a predetermined value in any of the time frames as in the second embodiment. Furthermore, the correlation may be created using, as the objects to be measured, subjects whose average value of the differences between the minimum value of the blood glucose level and the maximum value of the blood glucose level (Range) in a plurality of time frames is equal to or smaller than a predetermined value.

The correlation may be created using not only the difference between the minimum value of the blood glucose level and the maximum value of the blood glucose level (Range) but also data of objects to be measured as determined based on other trends of the blood glucose level. For example, in view of the fact that a subject who is chronically in a hyperglycemic state has a higher median value of the blood glucose level (Median) than a subject who is not chronically in a hyperglycemic state, the correlation may be created using, as the objects to be measured, subjects whose median value of the blood glucose level (Median) is equal to or smaller than a predetermined value. The correlation may be created using, as the objects to be measured, subjects whose difference between the median value of the blood glucose level (Median) and the maximum value of the blood glucose level is equal to or smaller than a predetermined value. Furthermore, the correlation may be created using objects to be measured as determined based on both the difference between the minimum value of the blood glucose level and the maximum value of the blood glucose level (Range) and the median value of the blood glucose level (Median) described above.

Generally, if a subject has a fasting blood glucose level of 126 mg/dL or higher, the subject is regarded as diabetic. Thus, subjects having a fasting blood glucose level of 126 mg/dL or higher may be excluded from the objects to be measured when creating the correlation. Furthermore, if a subject has a hemoglobin value (HbAlc) of 6.5% or higher, the subject is regarded as diabetic. Thus, subjects having a hemoglobin value (HbAlc) of 6.5% or higher may be excluded from the objects to be measured when creating the correlation.

### Third Embodiment

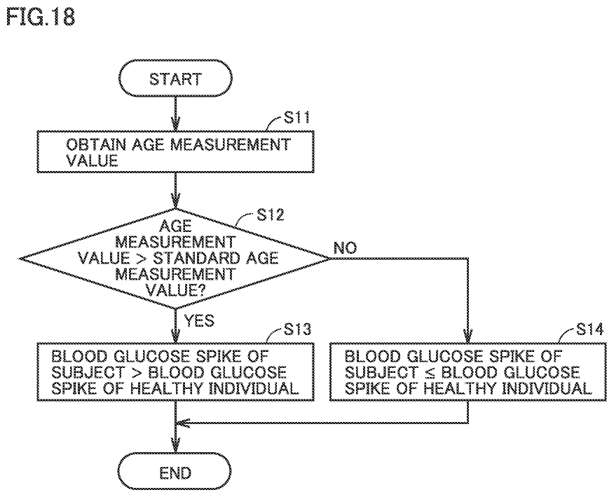

Estimation system 1 and estimation device 50 according to a third embodiment will be described with reference to FIG. 18. Estimation system 1 and estimation device 50 according to the third embodiment will be described below in terms of differences relative to estimation system 1 and estimation device 50 according to each of the first embodiment and the second embodiment, and the same portions as those of estimation system 1 and estimation device 50 according to each of the first embodiment and the second embodiment may not be described.

FIG. 18 is a flowchart of a determination process performed by estimation device 50 according to the third embodiment. Process steps (hereinafter abbreviated as “S”) shown in FIG. 18 are implemented by computing device 510 executing estimation program 521.

As shown in FIG. 18, estimation device 50 obtains an AGE measurement value of a subject from AGE measurement device 10 (S11). Estimation device 50 compares the obtained AGE measurement value of the subject with a standard AGE measurement value of a healthy individual (S12). Estimation device 50 stores the standard AGE measurement value of the healthy individual in storage device 520 in advance.

When the AGE measurement value of the subject is larger than the standard AGE measurement value of the healthy individual (YES in S12), estimation device 50 determines that a blood glucose spike frequency of the subject is higher than a blood glucose spike frequency of the healthy individual (S13). When the AGE measurement value of the subject is equal to or smaller than the standard AGE measurement value of the healthy individual (NO in S12), on the other hand, estimation device 50 determines that the blood glucose spike frequency of the subject is equal to lower than the blood glucose spike frequency of the healthy individual (S13).

As described above, estimation device 50 can determine the blood glucose spike frequency of the subject by comparing the AGE measurement value of the subject obtained by measurement device 10 with the standard AGE measurement value of the healthy individual. As a result, estimation device 50 can estimate the blood glucose spike frequency based on the AGE measurement value of the subject without performing the continuous glucose monitoring of the subject, thereby allowing the user to recognize the blood glucose spike frequency while suppressing the burden on the subject.

Although estimation system 1 and estimation device 50 according to each of the first to third embodiments have been described, the configurations and functions (processes) included in estimation system 1 and estimation device 50 according to each embodiment can be combined.

In estimation system 1 and estimation device 50 according to each of the first to third embodiments, the AGEs of the subject are used to estimate the blood glucose spike frequency of the subject. Therefore, the AGEs of the subject function as a marker for estimating the blood glucose spike frequency of the subject.

### <Aspects>

It will be understood by those skilled in the art that the plurality of exemplary embodiments described above are specific examples of aspects below.

(Clause 1) An estimation method according to an aspect includes, as processing to be performed by a computing device: obtaining a measurement value of advanced glycation end products of a subject; and estimating, using a correlation between a measurement value of advanced glycation end products and a blood glucose spike frequency prepared in advance, a blood glucose spike frequency of the subject based on the measurement value of advanced glycation end products of the subject obtained in the obtaining.

According to the estimation method described in Clause 1, the subject does not need to perform continuous glucose monitoring, and a user can readily recognize the blood glucose spike frequency using the advanced glycation end products of the subject while suppressing a burden on the subject.

(Clause 2) In the estimation method according to Clause 1, the correlation is generated based on a measurement value of advanced glycation end products of each of a plurality of subjects and a blood glucose spike frequency of each of the plurality of subjects.

According to the estimation method described in Clause 2, the blood glucose spike frequency of the subject is estimated using the correlation generated based on a measurement value of advanced glycation end products of each of a plurality of subjects and a blood glucose spike frequency of each of the plurality of subjects, so that the blood glucose spike frequency of the subject can be readily estimated using the correlation prepared in advance.

(Clause 3) In the estimation method according to Clause 1 or 2, the correlation is a regression line generated based on the measurement value of advanced glycation end products of each of the plurality of subjects and the blood glucose spike frequency of each of the plurality of subjects. The estimating includes estimating, using the regression line, the blood glucose spike frequency of the subject based on the measurement value of advanced glycation end products of the subject obtained in the obtaining.

According to the estimation method described in Clause 3, the blood glucose spike frequency of the subject can be readily estimated using the regression line generated based on the measurement value of advanced glycation end products of each of the plurality of subjects and the blood glucose spike frequency of each of the plurality of subjects.

(Clause 4) In the estimation method according to any one of Clauses 1 to 3, the blood glucose spike frequency of each of the plurality of subjects is obtained by calculating, for measurement values of a blood glucose level of each of the plurality of subjects obtained at predetermined intervals in a predetermined period, a number of the measurement values of the blood glucose level exceeding a predetermined value.

According to the estimation method described in Clause 4, of the measurement values of the blood glucose level obtained at predetermined intervals in a predetermined period, the number of the measurement values exceeding a predetermined value is used as the blood glucose spike frequency for generating the correlation, so that the subject can estimate the blood glucose spike frequency in the predetermined period based on the measurement value of advanced glycation end products.

(Clause 5) In the estimation method according to any one of Clauses 1 to 4, the measurement value of advanced glycation end products of each of the plurality of subjects is obtained in at least one measurement in the predetermined period.

According to the estimation method described in Clause 5, the measurement value of advanced glycation end products in at least one measurement in the predetermined period is used as the measurement value of advanced glycation end products for generating the correlation, so that the subject can readily estimate the blood glucose spike frequency by at least one measurement of advanced glycation end products in the predetermined period in a similar manner.

(Clause 6) In the estimation method according to any one of Clauses 1 to 5, the plurality of subjects are subjects suspected of being potential diabetics determined based on a comparison of a value relating to blood glucose level that differs between a subject affected with diabetes and a subject not affected with diabetes with a predetermined value.

The estimation method described in Clause 6 uses the correlation created using, as the objects to be measured, the subjects identified based on a comparison of a value relating to blood glucose level that differs between a subject affected with diabetes and a subject not affected with diabetes with a predetermined value, and can therefore exclude subjects who are chronically in a hyperglycemic state and subjects in which blood glucose spikes do not occur, for example, from the objects to be measured for the correlation. As a result, the blood glucose spike frequency of the subject can be estimated using the correlation created using potential diabetics as the objects, which allows for estimation of whether or not the subject is a potential diabetic.

(Clause 7) In the estimation method according to any one of Clauses 1 to 6, the value relating to blood glucose level includes at least one of a difference between a minimum value of blood glucose level and a maximum value of blood glucose level, a median value of blood glucose level, and a difference between the median value of blood glucose level and the maximum value of blood glucose level.

According to the estimation method described in Clause 7, the user can exclude subjects who are chronically in a hyperglycemic state and subjects in which blood glucose spikes do not occur from the objects to be measured for the correlation.

(Clause 8) In the estimation method according to any one of Clauses 1 to 7, the measurement value of advanced glycation end products of the subject is obtained by non-invasive measurement.

According to the estimation method described in Clause 8, the subject can readily estimate the blood glucose spike frequency by non-invasive measurement of advanced glycation end products.

(Clause 9) An estimation program according to an aspect causes a computing device to perform: obtaining a measurement value of advanced glycation end products of a subject; and estimating, using a correlation between a measurement value of advanced glycation end products and a blood glucose spike frequency prepared in advance, a blood glucose spike frequency of the subject based on the measurement value of advanced glycation end products of the subject obtained in the obtaining.

According to the estimation program described in Clause 9, the subject does not need to perform continuous glucose monitoring, and a user can readily recognize the blood glucose spike frequency using the advanced glycation end products of the subject while suppressing a burden on the subject.

(Clause 10) An estimation system according to an aspect includes: a measurement device that measures advanced glycation end products of a subject; an estimation device that estimates, using a correlation between a measurement value of advanced glycation end products and a blood glucose spike frequency prepared in advance, a blood glucose spike frequency of the subject based on a measurement value of the advanced glycation end products of the subject measured by the measurement device; and a display device that displays viewing information based on the blood glucose spike frequency of the subject estimated by the estimation device.

According to the estimation system described in Clause 10, the subject does not need to perform continuous glucose monitoring, and a user can readily recognize the blood glucose spike frequency using the advanced glycation end products of the subject while suppressing a burden on the subject.

(Clause 11) A determination method according to an aspect includes, as processing to be performed by a computing device: obtaining a measurement value of advanced glycation end products of a subject; comparing the measurement value of advanced glycation end products of the subject obtained in the obtaining with a standard measurement value of advanced glycation end products of a healthy individual; and when the measurement value of advanced glycation end products of the subject is larger than the standard measurement value of advanced glycation end products, determining that a blood glucose spike frequency of the subject is higher than a blood glucose spike frequency of the healthy individual.

According to the determination method described in Clause 11, the subject does not need to perform continuous glucose monitoring, and a user can readily recognize the blood glucose spike frequency using the advanced glycation end products of the subject while suppressing a burden on the subject.

(Clause 12) An estimation marker according to an aspect includes advanced glycation end products for estimating a blood glucose spike frequency.

According to the determination method described in Clause 12, the subject does not need to perform continuous glucose monitoring, and a user can readily recognize the blood glucose spike frequency using the advanced glycation end products of the subject while suppressing a burden on the subject.

### REFERENCE SIGNS LIST

1 estimation system; 10 measurement device; 11 measurement unit; 12, 390 display; 13 communication unit; 30 display device; 31 home screen; 50 estimation device; 311, 312, 313, 314 image; 510 computing device; 520 storage device; 521 estimation program; 522 user identification information; 523 viewing information; 524 advice information; 530 communication device.

1. An estimation method for estimating a blood glucose spike frequency of a subject by a computing device, the estimation method comprising, as processing to be performed by the computing device:
obtaining a measurement value of advanced glycation end products of the subject; and
estimating, using a correlation between a measurement value of advanced glycation end products and a blood glucose spike frequency prepared in advance, a blood glucose spike frequency of the subject based on the measurement value of advanced glycation end products of the subject obtained in the obtaining, wherein
the correlation is generated based on a measurement value of advanced glycation end products of each of a plurality of subjects and a blood glucose spike frequency of each of the plurality of subjects, and
the plurality of subjects do not include a subject who is chronically in a hyperglycemic state determined based on measurement values of a blood glucose level in a predetermined period.

2. The estimation method according to claim 1, wherein
the subject who is chronically in a hyperglycemic state is determined based on a comparison of a difference between a minimum value of the blood glucose level and a maximum value of the blood glucose level in a particular time frame included in the predetermined period with a predetermined value.

3. The estimation method according to claim 1, wherein
the correlation is a regression line generated based on the measurement value of advanced glycation end products of each of the plurality of subjects and the blood glucose spike frequency of each of the plurality of subjects, and
the estimating includes estimating, using the regression line, the blood glucose spike frequency of the subject based on the measurement value of advanced glycation end products of the subject obtained in the obtaining.

4. The estimation method according to claim 1, wherein
the blood glucose spike frequency of each of the plurality of subjects is obtained by calculating, for measurement values of a blood glucose level of each of the plurality of subjects obtained at predetermined intervals in the predetermined period, a number of the measurement values of the blood glucose level exceeding a predetermined value.

5. The estimation method according to claim 4, wherein
the measurement value of advanced glycation end products of each of the plurality of subjects is obtained in at least one measurement in the predetermined period.

6. The estimation method according to claim 1, wherein
the plurality of subjects are subjects suspected of being potential diabetics determined based on a comparison of a value relating to blood glucose level that differs between a subject affected with diabetes and a subject not affected with diabetes with a predetermined value.

7. The estimation method according to claim 6, wherein
the value relating to blood glucose level includes at least one of
a difference between a minimum value of blood glucose level and a maximum value of blood glucose level,
a median value of blood glucose level, and
a difference between the median value of blood glucose level and the maximum value of blood glucose level.

8. The estimation method according to claim 1, wherein
the measurement value of advanced glycation end products of the subject is obtained by non-invasive measurement.

9. A non-transitory computer-readable storage medium storing an estimation program for estimating a blood glucose spike frequency of a subject, the estimation program causing a computing device to perform:
obtaining a measurement value of advanced glycation end products of the subject; and
estimating, using a correlation between a measurement value of advanced glycation end products and a blood glucose spike frequency prepared in advance, a blood glucose spike frequency of the subject based on the measurement value of advanced glycation end products of the subject obtained in the obtaining, wherein
the correlation is generated based on a measurement value of advanced glycation end products of each of a plurality of subjects and a blood glucose spike frequency of each of the plurality of subjects, and
the plurality of subjects do not include a subject who is chronically in a hyperglycemic state determined based on measurement values of a blood glucose level in a predetermined period.

10. An estimation system for estimating a blood glucose spike frequency of a subject, comprising:
a measurement device that measures advanced glycation end products of the subject;
an estimation device that estimates, using a correlation between a measurement value of advanced glycation end products and a blood glucose spike frequency prepared in advance, a blood glucose spike frequency of the subject based on a measurement value of the advanced glycation end products of the subject measured by the measurement device; and
a display device that displays viewing information based on the blood glucose spike frequency of the subject estimated by the estimation device, wherein
the correlation is generated based on a measurement value of advanced glycation end products of each of a plurality of subjects and a blood glucose spike frequency of each of the plurality of subjects, and
the plurality of subjects do not include a subject who is chronically in a hyperglycemic state determined based on measurement values of a blood glucose level in a predetermined period.

11-12. (canceled)
